(19) United States
(12) Patent Application Publication
(10) Pub. No.: US 2020/0316339 A1
(43) Pub. Date: Oct. 8, 2020

(54) CATHETER ASSEMBLY WITH LOW AXIAL SLIDING FRICTION

(71) Applicant: KONINKLIJKE PHILIPS N.V., Eindhoven (NL)

(72) Inventor: Steven Ernest Franklin, Eindhoven (NL)

(21) Appl. No.: 16/908,797

(22) Filed: Jun. 23, 2020

Related U.S. Application Data
(63) Continuation of application No. 15/747,538, filed on Jan. 25, 2018.

(30) Foreign Application Priority Data
Aug. 5, 2015 (EP) 15179821.2

(51) Int. Cl. A61M 25/00; A61B 5/00; A61M 25/09; A61B 34/20; A61B 5/06

(52) U.S. Cl. CPC A61M 25/0043; A61B 5/6852; A61M 25/09041; A61B 34/20; A61B 5/06; A61B 2034/2063; A61M 2025/0004; A61M 2025/0062; A61B 5/062; A61B 2034/2051; A61B 2034/2061; A61B 5/065

(57) ABSTRACT

Sliding friction between a catheter and a guiding core is reduced by a rotating inner catheter member (12), introduced between the guiding core (13) and an outer catheter member (11). The inner catheter member (12) is rotated in the outer catheter member (11) by a motor (14), which may be controlled by a control unit (2). A lower catheter friction with respect to the guidewire allows the physician improved assessment of the forces resulting from collisions of the outer catheter member (11) and the vascular wall.

### CROSS-REFERENCE TO RELATED APPLICATIONS

This application is a continuation of U.S. application Ser. No. 15/747,538, filed on Jan. 25, 2018, which is the U.S. National Phase application under 35 U.S.C. § 371 of International Application No. PCT/EP2016/067476, filed on Jul. 22, 2016, which claims the benefit of European Application Serial No. 15179821.2, filed Aug. 5, 2015. These applications are hereby incorporated by reference herein.

### FIELD OF THE INVENTION

The invention relates to a catheter and method for insertion over a guidewire through a patient's vasculature. The invention further relates to a system comprising the catheter and the guidewire.

### BACKGROUND OF THE INVENTION

Catheters are used for diagnosing various anatomical structures of living beings and for delivering therapy by accessing anatomical structures through natural cavities of the body. A particular group of catheter based interventions is represented by the diagnosing and treating of vascular diseases, where the catheter is advanced through the vasculature to a specific site with the aid of an imaging technique able to show the position of the catheter with respect to the diseased segment of the vessel. A guidewire is used for guiding the catheter through the bends, loops, and branches of the vasculature. A method of using a guidewire to direct the catheter through the torturous vasculature involves the use of a torqueable guidewire which is first guided to the targeted site of the vessel in the anatomical structure by repetitive rotating and advancing motions, then in a second step the catheter is advanced along the guidewire to reach itself the targeted site. Typically, for accessing remote body regions such as peripheral vasculature or accessing soft tissue such as for instance brain and liver, the guidewire and the catheter are advanced in a repetitively alternating pattern comprising the steps of advancing the guidewire along a distance in the vessel, holding the guidewire in place, and then advancing the catheter along the guidewire until it reaches the distal portion of the guidewire. The resistance felt by the clinician when advancing the catheter along the guidewire plays an important part of the catheterization procedure and it contributes significantly to the outcome of the procedure. One of the mechanisms responsible for the resistance in advancing the catheter towards the site of interest is the friction between the catheter and the guidewire, the other one is the friction between the catheter and the wall of the vasculature. A lower catheter friction with respect to the guidewire allows the physician to assess better the forces resulting from collisions of the catheter with the vascular wall since the friction experienced by the clinician will depend much more on the catheter-tissue friction and much less on the catheter-guidewire friction. Misinterpretation of the resistance felt by the physicians in advancing catheters along the guidewire through complex vasculature can easily lead to clinical complications related to vessel wall trauma. Typical technical solutions aiming for low friction guidewire-catheter combination include: increasing the pitch of the guidewire coiling for reducing the contact surface between the guidewire and catheter, using low friction coating on the contacting surface of the guidewire and/or on that of the catheter.

U.S. Pat. No. 8,518,099 B2 presents various solutions for reducing friction between an outer sheath and an inner carrier catheter slidably disposed within the outer sheath. A plurality of implant support structures are formed between adjacent internal longitudinal voids on the inner surface of the outer sheath in order to reduce the contact surface between the outer sheath and the inner carrier catheter. Tight angles when negotiating sharp bends in the vasculature give higher pressures to the internal support structures, which may partially neutralize the reduction of sliding friction due to contact area diminution.

US 20020016624 A1 discloses a catheter system for removing stenotic material from within previously stented region of a patient's vasculature. The catheter system includes an inner catheter shaft having a stenotic material removal mechanism and a guidewire lumen for introduction of the catheter system over a guidewire. The catheter system further comprises an outer catheter tube. A motor drive unit coupled to the proximal end of the inner catheter shaft rotates and/or axially translates the stenotic material removal mechanism of the inner catheter shaft for removing stenotic material from within the stented vessel. The drive motor, which may be a low speed motor or gear motor that operates at a speed from 500 to 2000 rpm or a high speed motor or a turbine that operates at a speed from 2000 to 150000 rpm, rotates the stenotic material removal mechanism of the inner catheter shaft at the respective speed.

### SUMMARY OF THE INVENTION

It is an object of the invention to provide a catheter assembly with reduced catheter sliding friction with respect to its guiding core.

According to the invention, this object is realized by a catheter assembly comprising:

a flexible outer catheter member having a proximal end, a distal end, and a lumen extending from the proximal end to the distal end;

a flexible inner catheter member disposed rotatable within the lumen of the outer catheter member, the inner catheter member having a proximal end, a distal end, and a lumen extending from the proximal end to the distal end;

a motor coupled to the inner catheter member;

wherein the lumen of the inner catheter member is adapted to receive a movable guidewire;

wherein the motor is configured to rotate the inner catheter member with respect to the outer catheter member at a rotational speed below 500 rpm, such as to reduce sliding friction of the guidewire with respect to the outer catheter member when the guidewire is received by the lumen of the inner catheter member.

The sliding friction force is reduced by introduction of a rotating intermediate inner catheter member between the guiding core and the outer catheter member. Furthermore, the dependence of the sliding friction force on catheter bending is reduced, resulting in an improved predictability and reproducibility of the sliding friction force range encountered in catheterization procedures. When sliding friction force is reduced between catheter and its guiding core, then the forces resulting from collisions of the catheter with the vasculature walls dominate, and the physician is able to rely on the fact that the resistance to advancing the catheter in the vasculature originates from catheter-vessel wall interaction rather than from internal friction between the components of the catheter assembly.

In a further embodiment of the catheter assembly, the distal end of the guidewire comprises a sensor for measuring at least one of a pressure, a flow, and an electrical signal. Measurements at the distal end of the guiding core can improve positioning the catheter with respect to a target site.

In yet a further embodiment of the catheter assembly, the outer catheter member also comprises a sensor for measuring at least one of a pressure, a flow, and an electrical signal. By enabling functional measurements based on differential pressure, flow or electrical signal measurements the physician can identify various anomalies in the vasculature, such as stenosis of a vessel.

In an embodiment of the catheter assembly, the outer catheter member and the guidewire comprise position sensors. The position sensors can give information on their positions with respect to each other, and the physician can decide whether to advance different components of the catheter assembly further relative to each other. The position sensors may be based on electromagnetic, ultrasound, or optical technology, or a combination of these.

In another aspect of the invention a system is presented, comprising the catheter assembly and a control unit for controlling a rotational speed with which the motor rotates the inner catheter member. The controlling unit is connected to the motor for rotating the inner catheter member of the catheter assembly, such that the physician can control the rotational speed of the inner catheter member, and thus the sliding friction between the guidewire and the catheter assembly, according to the phase of the catheterization procedure.

In another embodiment, the system further comprises a position tracking unit, wherein the control unit is configured to determine the position of the guidewire and the position of the outer catheter member based on signals received from the position tracking unit and/or from the position sensors. The position tracking of the different components of the catheter assembly improves advancing the catheter to a targeted site in the vasculature.

In yet a further embodiment of the system, the control unit is configured to determine a sliding velocity of the guidewire with respect to the outer catheter member, and the control unit is further configured to adapt the rotational speed of the inner catheter member depending on the sliding velocity of the guidewire with respect to the outer catheter member. In certain phases of the catheterization procedure it is advantageous to relate the rotational speed of the inner catheter member to the sliding velocity. Such example is when stability of functional measurements is of high importance. A discontinuation in advancing the outer catheter member with respect to the guidewire may trigger an adjustment of the rotational speed of the inner catheter member for the duration of the measurements.

In another embodiment of the system, the guidewire and the outer catheter member of the catheter assembly comprise optical sensors for shape and position determination of the catheter assembly, wherein the control unit is configured to send optical signals to the optical sensors in the guidewire and in the outer catheter member of the catheter assembly, and the control unit is further configured to determine a position of the guidewire and a position of the outer catheter member based on the signals received from the optical sensors. Optical fibers may be used for position sensing of the distal end of the guidewire with respect to that of the outer catheter member. Such system has the advantage that an external position tracking unit is not required. Additionally, the system can determine the shape of the catheter assembly.

In yet a further aspect of the invention, a method for reducing friction in a catheter assembly is presented, the method comprising:

providing rotation of the inner catheter member with respect to the outer catheter member by a motor;

providing a sliding motion between the guidewire and the outer catheter member;

wherein the rotation of the inner catheter member is at a rotational speed below 500 rpm, such as to reduce sliding friction of the guidewire (13) with respect to the outer catheter member (11) when the guidewire (13) is received by the lumen of the inner catheter member (12).

In another embodiment, the method may additionally comprise the steps of:

providing position signals of the outer catheter member and the guidewire; ascertaining a relative sliding velocity of the outer catheter member with respect to the guidewire;

adapting a rotational speed of the inner catheter member based on the relative sliding velocity.

When a physician advances the catheter to the desired position and the relative motion between the outer catheter member and the guidewire stops for a predetermined duration, then the method allows automatic adjustment of the rotational speed of the inner catheter member to a standby rotational regime.

Additional aspects and advantages of the invention will become more apparent from the following detailed description, which may be best understood with reference to and in conjunction with the accompanying drawings.

### BRIEF DESCRIPTION OF THE DRAWINGS

In the drawings:

FIG. 1 shows schematically and exemplarily an embodiment of a catheter assembly according to the invention.

FIG. 2 shows schematically and exemplarily a system comprising the catheter assembly, a control unit, and an external tracking unit.

FIG. 3A shows schematically the friction force at a contact point between a sliding core and a static inner catheter member.

FIG. 3B shows schematically the friction force at a contact point between a sliding core and a rotating inner catheter member.

FIG. 4 shows schematically the bending of the catheter assembly in the experimental setup.

FIG. 5 shows experimental results and axial friction force estimation from a theoretical model.

FIG. 6 shows schematically and exemplarily an embodiment of the catheter assembly with integrated measurement sensors.

FIG. 7 shows schematically and exemplarily an embodiment of the catheter assembly with integrated position sensors.

FIG. 8 shows schematically and exemplarily an embodiment of a catheter assembly with integrated optical fibers for shape and position sensing.

FIG. 9 shows schematically and exemplarily a method for reduction of axial sliding friction in a catheter assembly according to the invention.

FIG. 10 shows experimental results of axial friction force measurements for rotation speeds up to 1000 rpm.

FIG. 11 shows axial friction force estimation from a theoretical model for rotation speeds up to 1000 rpm.

### DETAILED DESCRIPTION OF EMBODIMENTS

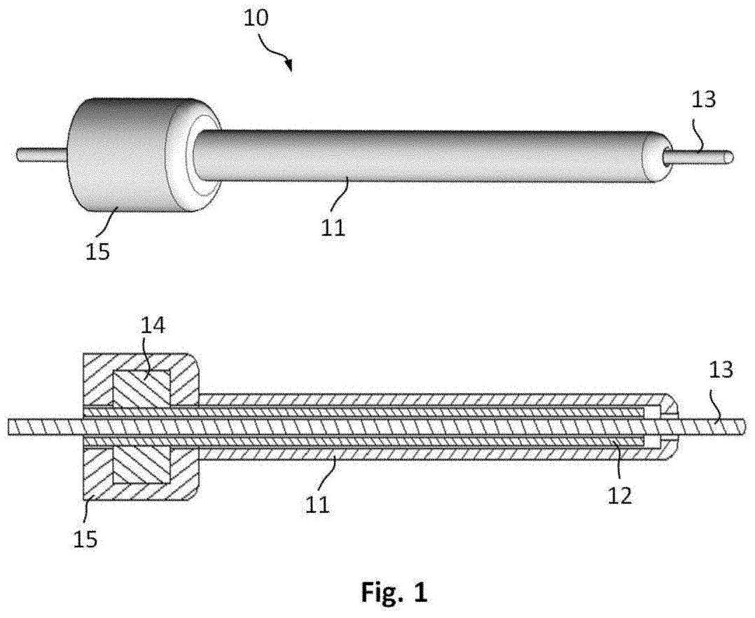

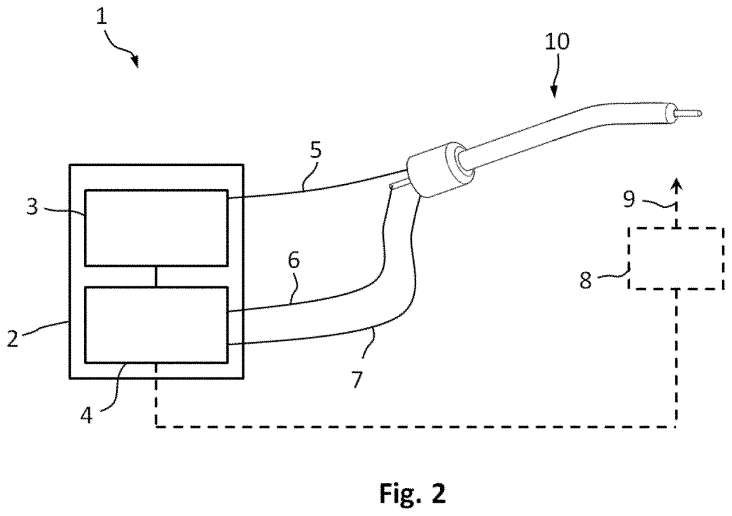

A schematic and exemplary embodiment of a catheter assembly 10 according to the invention is presented in FIG. 1. The catheter assembly 10 is suitable for accessing peripheral vasculature in remote body regions, and it comprises a hollow flexible outer catheter member 11, a flexible inner catheter member 12 disposed rotatable within the lumen of the outer catheter member 11, a flexible core 13 arranged movable within the lumen of the inner catheter member, and a motor 14 disposed in a handgrip 15 at the proximal end of the catheter assembly 10. The handgrip 15 may be a separate part from the outer catheter member 11 and fixed to it, or alternatively the two parts may be made from the same material and may form together the outer catheter member. The motor 14 is coupled to the inner catheter member 12, and is configured to rotate the inner catheter member 12 with respect to the outer catheter member 11. In an embodiment, the motor 14 may have energy supply by means of batteries, and it may be controlled by a switch integrated into the handgrip. In an alternative embodiment, illustrated schematically in FIG. 2, the energy to the motor 14 is provided by an energy supply 3 in the control unit 2 of the system 1. The energy may be supplied by the connection 5 between the energy supply 3 and the motor 14, or alternatively by wireless transmission.

A method to bring a catheter to a remote site in the vasculature of the body is to use a core 13, typically a flexible guidewire that is first guided by the physician to the targeted site of the vessel in the anatomical structure by repetitive rotating and sliding motions. In a second step, the guidewire is kept in position and the catheter is advanced along the guidewire to reach itself the targeted site. In typical operation of the catheter assembly 10, the outer catheter member 11 fixed to the handgrip 15 is held by the physician and it is handled carefully not to create excessive trauma by its manipulation through the vasculature. Therefore, the role of the outer catheter member 11 is protection of the vessel walls during operation, when the motor 14 generates revolution of the inner catheter member 12 with respect the outer catheter member 11. A direct exposure of the spinning internal catheter member 12 could damage the vessel walls. The relative motion between the guiding core 13 and the outer catheter member 11 consists of sliding at a rate controlled by the physician in order to advance the catheter with respect to the core towards the targeted site, and slight occasional torque inherent to the experience of the practicing physician. Typically, physicians use torque when they feel resistance in advancing the catheter, which partly originates from catheter friction with respect to the guidewire and partly from the catheter interaction with the vessel walls. By reducing the internal friction between the catheter and the guidewire, the torque that physicians would need to apply, in order to move the outer catheter member 11 in contact with the vessel walls with respect to the core 13 guidewire or vice versa, would be less.

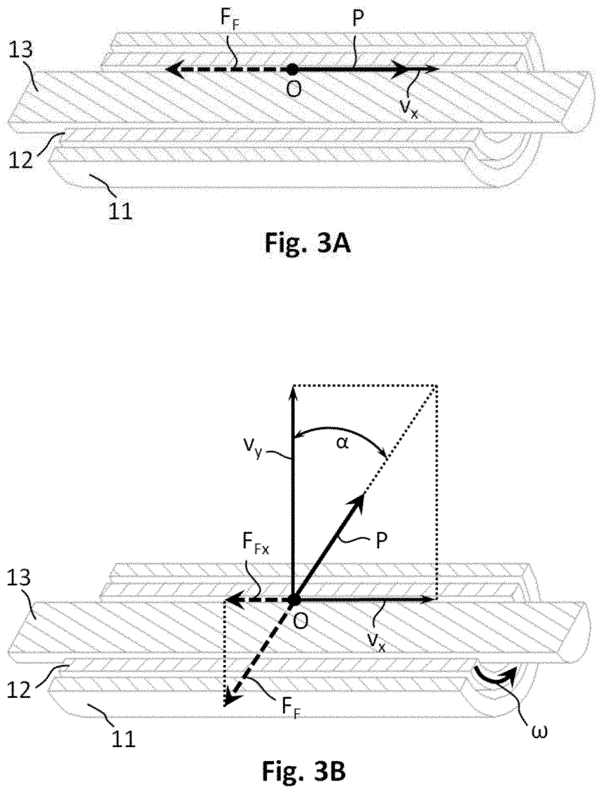

FIGS. 3A and 3B illustrate schematically the theoretical situations for advancing a core 13 at a velocity vₓ with respect to the outer catheter member 11. The assumption is that in point O the core 13 contacts the inner catheter member 12. In FIG. 3A the inner and the outer catheter members 12, 11 are stationary, and the force for sliding the core 13 with respect to the inner and outer catheter members 12, 11 is denoted with P. The friction force F_{F} in case of constant sliding velocity is equal to the force P. FIG. 3B shows a different situation, where the inner catheter member 12 revolves at an angular velocity ω with respect to the outer catheter member 11 and with respect to the core 13, whereas the core 13 slides at the velocity vₓ with respect to the outer catheter member 11. The revolution of the inner catheter member results in a tangential velocity in point O, according to:

v_{y}=rω (Eq. 1)

where r is the inner radius of the inner catheter member 12.

When both velocities, the tangential velocity v_{y} resulting from the revolution of the inner catheter member and the axial velocity vₓ of the sliding, are constant, then the direction of the force P will be deflected in a direction defined by the combination of the two velocity vectors, forming an angle a with respect to the direction of the tangential velocity.

α=tan⁻¹(vₓ/v_{y}) (Eq. 2)

Although, similar to the previous case, the friction force F_{F} is equal to the force P, the projection of the friction force on the longitudinal sliding axis F_{Fx} is considerably smaller.

F_{Fx}=F_{F} sin α (Eq. 3)

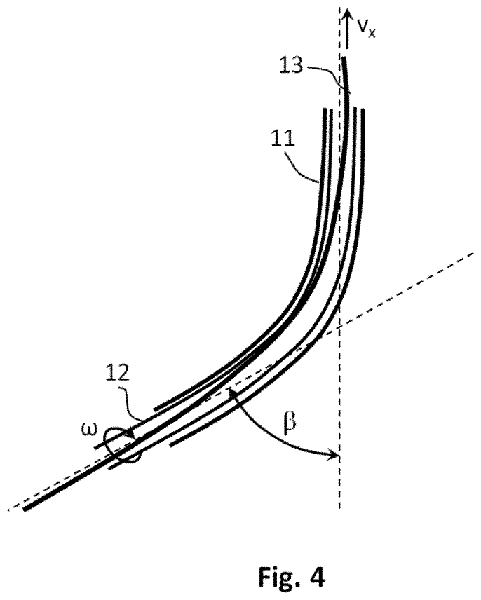

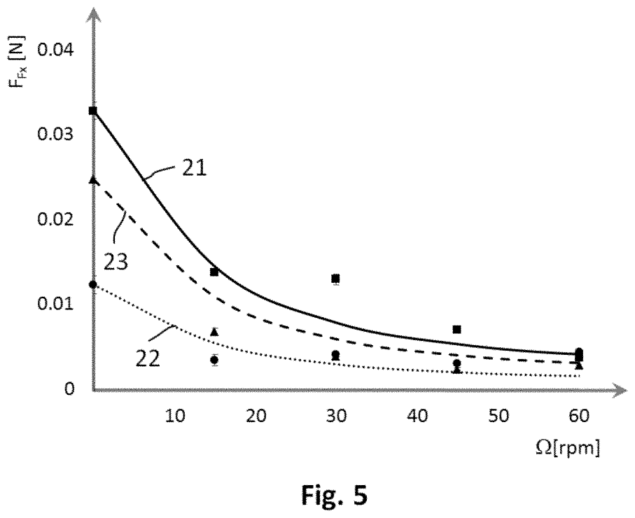

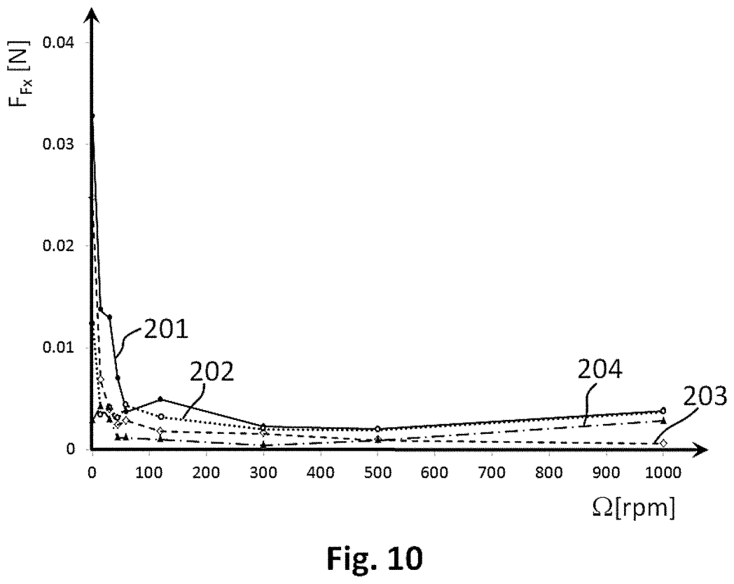

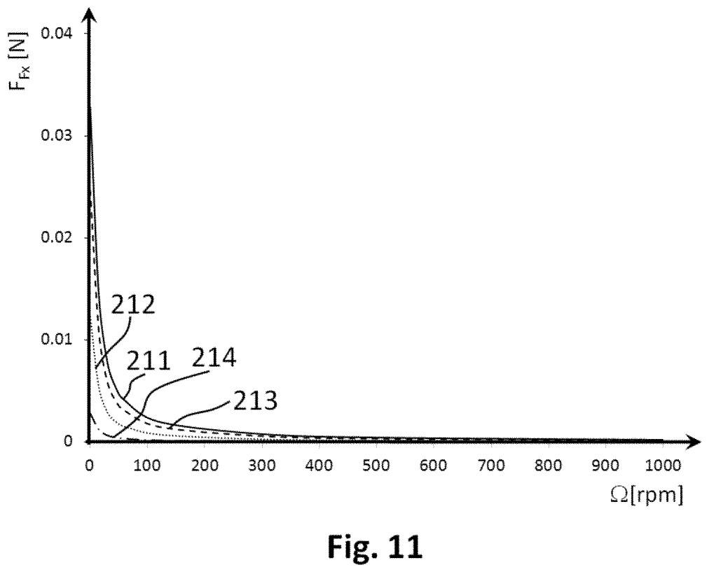

For a practical example a Pebax 7233 material with a length of 45 cm was chosen for the inner and the outer catheter members 12, 11. The internal diameter of the inner catheter member was 2.6 mm. A Terumo Radiofocus Guidewire made of nitinol alloy and coated with polyurethane jacket having an external diameter of 0.90 mm was used as core 13. An electric Zwick test machine fitted with a calibrated 20 Newton load cell was used to withdraw and advance the guidewire core 13 within the rotating inner catheter member 12 tube, schematically represented in FIG. 4. The friction force was measured throughout the experiment, while the sliding velocity vₓ was kept constant at 1 mm/s. The test was carried out for a range of bending angles β of the catheter assembly 10 in the interval of 0° to 135° and for a range of rotational speeds Ω of the inner catheter member 12 in the interval of 0 to 1000 revolutions per minute (rpm). Theoretically estimated results and experimentally measured results of axial friction force F_{Fx} dependence on rotation speed Ω are exemplarily presented in FIG. 5. Lines 21, 22, 23 represent the theoretical results for 0°, 20° and 135° bending angles β of the catheter assembly 10, whereas the experimental results are illustrated with squares, circles and triangles, corresponding to the same respective bending angles. The discrepancy between the measured values from the theoretically calculated ones may originate from occasional multiple contacts between the core 13 and inner catheter member 12, nevertheless the theoretical model gives a satisfactory estimation of the expected axial sliding friction force. At higher rotational speed than 60 rpm, the measured friction force remained relatively constant and it did deviate from the theoretical model, presumably due to bouncing of the core in the inner catheter member. Experiments showed that reduction of axial sliding friction force of the core with respect to the outer catheter member can be achieved at least up to a rotational speed of 1000 rpm and for bending angles up to 135°, thresholds which are merely attributed to the limitations of the test setup. Experimental axial friction force measurements are presented in FIG. 10 for selected discrete rotational speeds up to 1000 rpm. Lines 201, 202, 204 and 203 represent the axial friction force measurements for 0°, 20°, 90° and 135° bending angles β of the catheter assembly, respectively. Estimated axial friction force values 211, 212, 213, and 214 for rotational speeds up to 1000 rpm based on the theoretical model are presented in FIG. 11. A comparison of the experimental results with the theoretical model reveals a similar decreasing trend of the axial friction force with increasing rotation speeds up to about 500 rpm, whereas above 500 rpm the experimental results show an increasing trend of the axial friction force with increasing rotational speed (for bending angles β of the catheter assembly of 0°, 20°, 90°), a dissimilar trend from the results expected based on the theoretical model. As mentioned earlier, the divergence of the theoretical model from the experimental results is presumably generated by bouncing of the core in the inner catheter member. Since the mechanism is not fully understood why above rotational speeds of 500 rpm in most of the cases an increase of axial friction forces results in practice, it is recommended that the catheter assembly is used below rotational speeds of 500 rpm. The bouncing effect may generate undesired vibrations of the catheter assembly, potentially leading to damaging vessel walls during clinical practice. In an embodiment of the invention the rotational speed is chosen in the interval of 20 to 480 rpm, in order to ensure that the upper limit of rotational speeds is avoided at which in the practical experiments the unexpected bouncing effect was produced, and also for avoiding the too low rotational speed values for which there was still insufficient diminution of the axial friction force.

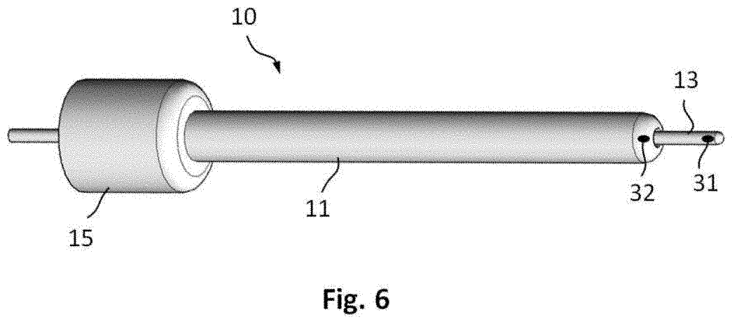

FIG. 6 shows an embodiment of the catheter assembly 10, wherein the distal end of the core 13 comprises at least a sensor 31 for measuring at least one of a force, a pressure, a flow, and an electrical signal. Correspondingly, the distal end of the outer catheter member 11 may comprise at least a sensor 32, for similar measurements. The measurement signals are transmitted from the sensors integrated into the catheter assembly 10 to a measurement unit 4 through connections 6 and 7, illustrated schematically in FIG. 2. The signals are processed in a processor of the measurement unit 4, to derive physiological parameters. The control unit 2 may comprise a user interface allowing the physician to select various physiological parameters that can be an indication of a clinical condition of a patient. Typical examples are the pressure difference measured by the sensors 31, 32 that relates to stenosis of a blood vessel, force exerted by the tissue on the sensor, a contact resistance, timing and amplitude of electrical signals that relate to irregularities in electrical activity of an organ, for instance heart.

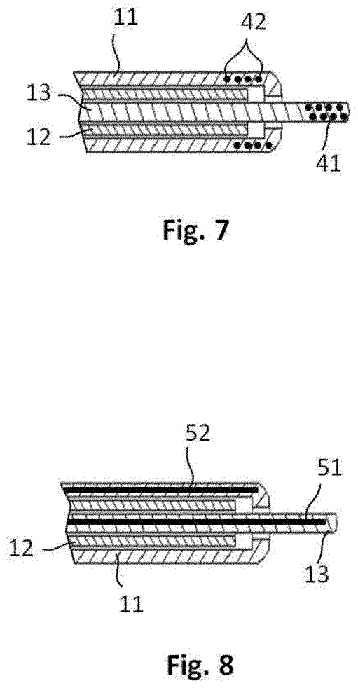

In FIG. 7 the cross section of the distal portion of another catheter assembly embodiment is presented schematically. The distal end of the core 13 and the distal end of the outer catheter member 11 comprise position sensors 41 and 42. The system 1 comprises a position tracking unit 8, connected to the control unit 2, as schematically illustrated in FIG. 2.

The position sensors 41, 42 may be electromagnetic sensors, and the position tracking unit 8 may be an electromagnetic field generating device. The electromagnetic field 9 is sensed by the position sensors 41, 42. The relative position of the sensors is derived from the strength of the measured electromagnetic field. In an alternative embodiment, the sensors 41, 42 may be ultrasound transducers, in which case the position tracking unit 8 is an external ultrasound transducer sending and/or receiving ultrasound signals 9. The measurement unit 4 receives signals form the position sensors 41, 42 and from the position tracking unit 8. The relative positions of the sensors are determined by computations performed by a processor of the control unit 2. From the variation of the relative positions of the sensors in time the processor can be configured to determine the sliding velocity vₓ of the core 13 with respect to the outer catheter member 11. The sliding velocity vₓ can be used by the control unit 2 to adapt the angular velocity ω of the inner catheter member 12 with respect to the outer catheter member 11. In case that the physician stops advancing either the outer catheter member 11 with respect to the core 13 or the core with respect to the outer catheter member, then the system can reduce the angular velocity to a standby setting or to stop the rotation of the inner catheter member. Typically, when physicians perform measurements with the sensors 31, 32 integrated into the catheter assembly 10, then they stop advancing the outer catheter member with respect to the core. By stopping the revolution of the inner catheter member in those instances, increased functional measurement stability can be obtained. For a practical implementation the time interval for determining an average sliding velocity of the core with respect to the outer catheter member may have a selectable duration, since the advancing technique of the catheter on a guidewire largely differs among practicing physicians.

In yet a further embodiment of the catheter assembly 10 schematically illustrated in FIG. 8, the core 13 and the outer catheter member 11 comprise optical sensors 51,52 for establishing the shapes and relative positions of the core 13 and the outer catheter member 11. Optical shape sensing is a technology that uses reflections from multiple optical fibers to measure local strain on each individual optical fiber. The strain on optical fibers is caused by tension due to bending of the structure in which they are integrated. The shape of the optical fibers is derived by processing optical reflection signals from within the optical fibers, received by a detector. The light source and the detector can be integrated in the control unit 2, and the optical fibers can easily be integrated into the core and the outer catheter member due to their small diameter. Once the position and the shape of the optical fibers are known, the relative positions and the shapes of the core and the outer catheter member can be computed by the processor of the control unit 2. The position tracking unit 8 can support the determination of the absolute position of the catheter assembly 10 in the vasculature of the patient. The position tracking unit may be an apparatus based on one of the following imaging modalities: ultrasound, fluoroscopy or magnetic resonance.

The control unit 2 comprises a computer, a computer-readable medium having stored a computer-executable program and a user interface. The computer program comprises program code means for determining a relative sliding velocity of the outer catheter member 11 with respect to the core 13 based on signals received from the position sensors 41, 42, and/or from the position tracking unit 8. It may further comprise program code means for adapting the angular velocity of the inner catheter member 12 based on the relative sliding velocity.

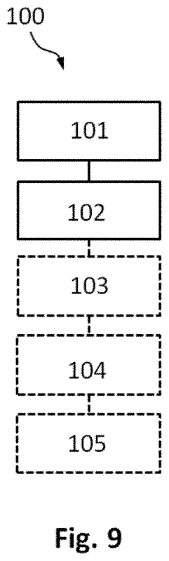

A typical use of the system 1 for reducing friction in the catheter assembly is illustrated schematically in FIG. 9, and it comprises the following steps: providing rotation ω of the inner catheter member 12 with respect to the outer catheter member 11 by a motor 14 in step 101, providing an axial sliding motion vₓ between the core 13 and the outer catheter member 11 in step 102. Additionally, the method 100 may comprise steps for automation of rotational speed adjustment of the inner catheter member 12 as function of the relative sliding velocity between the core 13 and the outer catheter member 11. In step 103 the position sensors are providing position signals of the outer catheter member 11 and the core 13 to the control unit 2, which are used in in step 104 for ascertaining the relative sliding velocity of the outer catheter member 11 with respect to the core 13, based on which in step 105 the control unit 2 is adapting the rotational speed of the inner catheter member 12. Physicians may set in the user interface a sampling time interval for which an average sliding velocity is determined, depending on their usual practice. They may also set a standby rotational speed when the average sliding velocity is in a certain interval. A particular example is when the physician stops sliding the outer catheter member with respect to the core in order to perform functional measurements. The rotational speed may be adjusted by the control unit 2 to nil, allowing stable measurements with the sensors integrated into the catheter assembly. Furthermore, the physician may set in the user interface to resume the procedure by a retraction of the catheter with respect to the core, which results in an average sliding velocity that triggers a restart of rotation of the inner catheter member 12. A slight retraction of the catheter with respect to the core is considered to be safer at a restart of the catheterization procedure than an immediate advancing motion of the catheter with respect to the guiding core, due to the fact that advancing would occur only after starting the rotation of the inner catheter member, hence already with a reduced friction between the core and catheter.

Other variations to the disclosed embodiments can be understood and effected by those skilled in the art in practicing the claimed invention, from a study of the drawings, the disclosure, and the appended claims.

A single unit or device may fulfill the functions of several items recited in the claims. The mere fact that certain measures are recited in mutually different dependent claims does not indicate that a combination of these measures cannot be used to advantage.

In the claims, the word “comprising” does not exclude other elements or steps, and the indefinite article “a” or “an” does not exclude a plurality.

Any reference signs in the claims should not be construed as limiting the scope.

1. A catheter assembly comprising:
a flexible outer catheter member having a proximal end, a distal end, and a lumen extending from the proximal end to the distal end, wherein the distal end of the flexible outer catheter member is configured to be inserted into a vessel of a patient;
a flexible inner catheter member disposed rotatable within the lumen of the outer catheter member, the inner catheter member having a proximal end, a distal end, and a lumen extending from the proximal end to the distal end, wherein the flexible outer catheter member extends over the distal end of the flexible inner catheter member; and
a motor coupled to the inner catheter member;
wherein the lumen of the inner catheter member is adapted to receive a movable guidewire;
wherein the motor is configured to rotate the inner catheter member, relative to the outer catheter member and the guidewire, at a non-zero rotational speed below 500 rpm to reduce sliding friction of the guidewire with respect to the outer catheter member when the guidewire is received by the lumen of the inner catheter member.

2. The catheter assembly according to claim 1, further comprising the guidewire.

3. The catheter assembly according to claim 2,
wherein the distal end of the guidewire comprises a sensor for measuring at least one of a force, a pressure, a flow, and an electrical signal.

4. The catheter assembly according to claim 1,
wherein the outer catheter member comprises a sensor for measuring at least one of a force, a pressure, a flow, and an electrical signal.

5. The catheter assembly according to claim 4, wherein the sensor is an ultrasound transducer.

6. The catheter assembly according to claim 2, wherein at least one of the outer catheter member and the guidewire comprise a position sensor.

7. The catheter assembly according to claim 6, wherein the position sensor is an electromagnetic sensor.

8. The catheter assembly according to claim 6, wherein the position sensor is an ultrasound transducer.

9. The catheter assembly according to claim 6, wherein the position sensor is an optical sensor.

10. A system comprising the catheter assembly according to claim 2 and a control unit for controlling a rotational speed with which the motor rotates the inner catheter member relative to the outer catheter member and the guidewire.

11. The system according to claim 10, wherein the guidewire and the outer catheter member of the catheter assembly comprise optical sensors for shape and position determination of the catheter assembly,
wherein the control unit is configured to send optical signals to the optical sensors in the guidewire and in the outer catheter member of the catheter assembly, and
wherein the control unit is further configured to determine a position of the guidewire and a position of the outer catheter member based on the signals received from the optical sensors.

12. The system according to claim 10, the system further comprising a position tracking unit in communication with the control unit,
wherein the catheter assembly comprises position sensors in the outer catheter member and in the guidewire, the position sensors in communication with the position tracking unit, and
wherein the control unit is configured to determine a position of the guidewire and a position of the outer catheter member based on signals received from the position tracking unit and/or from the position sensors.

13. The system according to claim 12,
wherein the control unit is configured to ascertain a sliding velocity of the guidewire relative to the outer catheter member, and
wherein the control unit is further configured to adapt the rotational speed of the inner catheter member depending on the sliding velocity of the guidewire relative to the outer catheter member.

14. A method for reducing friction in the catheter assembly according to claim 2, the method comprising:
providing a rotation of the inner catheter member relative to the outer catheter member by the motor;
providing a sliding motion between the guidewire and the outer catheter member;
wherein the rotation of the inner catheter member is at a non-zero rotational speed below 500 rpm to reduce sliding friction of the guidewire relative to the outer catheter member when the guidewire is received by the lumen of the inner catheter member.

15. The method according to claim 14, the method further comprising:
providing position signals of the outer catheter member and the guidewire;
ascertaining a relative sliding velocity of the outer catheter member relative to the guidewire;
adapting a rotational speed of the inner catheter member based on the relative sliding velocity.

16. The method according to claim 14, the method further comprising:
inserting the catheter assembly into the vessel such that providing the rotation and providing the sliding motion are performed while the catheter assembly is positioned within the vessel.
